(19) United States
(12) Patent Application Publication
(10) Pub. No.: US 2017/0327862 A1
(43) Pub. Date: Nov. 16, 2017

(54) ANALYTICAL METHOD FOR QUANITIFICATION OF VIABLE BACTERIA CONTAINED IN MICROBIOTA RESTORATION THERAPY (MRT) COMPOSITIONS

(71) Applicant: REBIOTIX, INC., Roseville, MN (US)

(72) Inventors: BETH ANNE-SZKUDLAREK BROWN, Plymouth, MN (US); JOSHUA ERICKSON, Champlin, MN (US); MAYA BURROUGHS, St. Paul, MN (US); IAN SINCLAIR, Lakeville, MN (US); COURTNEY R. JONES, Fridley, MN (US)

(21) Appl. No.: 15/594,487

(22) Filed: May 12, 2017

Related U.S. Application Data
(60) Provisional application No. 62/336,184, filed on May 13, 2016.

(51) Int. Cl. C12Q 1/06; C12Q 1/68; C12N 15/09; G01N 33/53; G01N 35/00; C12Q 1/00

(52) U.S. Cl. CPC C12Q 1/06; C12Q 1/686; C12Q 1/6851; G01N 33/53; C12N 15/09; C12Q 1/00; G01N 35/00

(57) ABSTRACT

Quantification of the viable bacterial microorganisms in a drug product for delivery via a gastro-nasal tube, an enema and/or a capsule or tablet. A molecular-based approach, such as PMA (propidium monazide)-qPCR (quantitative polymerase chain reaction) assays may be useful for quantification of viable bacteria. By utilizing PMA treatment in combination with qPCR, the number of viable bacterial cells in the sample can be determined.

### CROSS-REFERENCE TO RELATED APPLICATIONS

This application claims priority under 35 U.S.C. §119 to U.S. Provisional Application Ser. No. 62/336,184, filed May 13, 2016, the entirety of which is incorporated herein by reference.

### FIELD

The present disclosure pertains to methods for analyzing compositions for treating patients.

### BACKGROUND

A wide variety of compositions and methods have been developed for treating diseases and/or conditions of the digestive track. Of the known compositions and methods, each has certain advantages and disadvantages. There is an ongoing need to provide alternative compositions and methods for treating diseases and/or conditions of the digestive track.

### BRIEF SUMMARY

An illustrative process for quantifying viable bacterial microorganisms in a microbiota restoration therapy (MRT) drug substance may comprise preparing an MRT drug substance test sample from a MRT drug substance and preparing a control test sample from a control sample. The MRT drug substance test sample and the control test sample may be treated with propidium monazide (PMA) and then aliquoted into a plurality of individual wells of a test plate. The test plate may be exposed to a light for in the range of 10 minutes. DNA samples from the MRT drug substance test sample in each of the plurality of individual wells and the control test sample in each of the plurality of individual wells may be extracted. The DNA samples from the MRT drug substance test sample in each of the plurality of individual wells and the control test sample in each of the plurality of individual wells may be diluted with molecular grade water. A quantitative polymerase chain reaction (qPCR) mixture may be added to a plurality of wells of a second test plate and then the diluted DNA samples from the MRT drug substance test sample in each of the plurality of individual wells and the control test sample in each of the plurality of individual wells of the test plate may be added to the plurality of wells of the second test plate. The second test plate may then be centrifuged. The second test plate may be placed in a qPCR detection system and a thermocycler protocol initiated. The total colony forming units (CFU) per milliliter (mL) (CFU/mL) of each test sample in the plurality of individual wells of the second test plate may then be calculated.

In some embodiments, the microbiota restoration therapy (MRT) drug substance may be prepared from a human stool sample for delivery via an enema or gastro-nasal tube. The method for preparing the MRT drug substance may comprise collecting a fresh stool sample from a human donor, adding an amount of saline to the fresh stool sample, adding polyethylene glycol to the fresh stool sample at a concentration of about 30-90 g/L (or about 10-90 g/L), mixing the fresh stool sample, saline, and polyethylene glycol together to make a mixed composition and filtering the mixed composition and collecting the filtrate, wherein the filtrate defines the MRT drug substance.

In some embodiments, the microbiota restoration therapy (MRT) drug substance may be prepared from a human stool sample for oral delivery. The method for preparing the MRT drug substance may comprise collecting a human stool sample, purifying the human stool sample to form a purified sample, stabilizing the purified sample to form a stabilized sample, converting the stabilized sample to a solid, and adding one or more additives and/or excipients to the solid to form a treatment composition, wherein the treatment composition defines the MRT drug substance.

The above summary of some embodiments is not intended to describe each disclosed embodiment or every implementation of the present disclosure. The Figures and Detailed Description which follow more particularly exemplify these embodiments.

### BRIEF DESCRIPTION OF THE DRAWINGS

The disclosure may be more completely understood in consideration of the following detailed description of various embodiments of the disclosure in connection with the accompanying drawings, in which:

FIG. 1 is a flowchart depicting an overall process for manufacturing a standardized FMT composition; and,

FIG. 2 is a flowchart depicting further steps in a representative manufacturing process.

FIG. 3. is a flowchart depicting further steps in a representative manufacturing process.

FIG. 4 is a flowchart depicting an illustrative analytical method.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure. “Mammal” as used herein refers to any member of the class Mammalia, including, without limitation, humans and nonhuman primates such as chimpanzees, and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

The term “cryopreservation,” as used herein, refers to the process of cooling and storing biological cells, tissues, or organs at very low temperatures to maintain their viability. As a non-limiting example, cryopreservation can be the technology of cooling and storing cells at a temperature below the freezing point (e.g., 196 K) that permits high rates of survivability of the cells upon thawing.

The term “cryoprotectant,” as used herein, refers to a substance that is used to protect biological cells or tissues from the effects of freezing.

As used herein, the term “microbiota” can refer to the human microbiome, the human microbiota or the human gut microbiota. The human microbiome (or human microbiota) is the aggregate of microorganisms that reside on the surface and in deep layers of skin, in the saliva and oral mucosa, in the conjunctiva, and in the gastrointestinal, genito-urinary, or vaginal tracts of humans. The human microbiome is comprised of bacteria, fungi, and archaea. Some of these organisms perform tasks that are useful for the human host, but the function of the majority of the organisms that make up the human microbiome is unknown. Under normal circumstances, these microorganisms do not cause disease to the human host, but instead participate in maintaining health. Hence, this population of organisms is frequently referred to as “normal flora.”

The population of microorganisms living in the human gastrointestinal tract is commonly referred to as “gut flora” or “gut microbiota.” The microbial flora of the human gut encompasses a wide variety of microorganisms that aid in digestion, the synthesis of vitamins, and creating enzymes not produced by the human body.

The phrase “microbiota restoration therapy,” as used herein, refers to a composition which may include, but is not limited to, human fecal material containing viable gut flora from a patient or donor, a diluent, and a cryoprotectant. Additional compositions include equivalent freeze-dried and reconstituted feces or a “synthetic” fecal composition. The human fecal material is screened for the presence of pathogenic microorganisms prior to its use in the microbiota restoration therapy. The human fecal material is screened for the presence of Clostridium species including C. difficile, Norovirus, Adenovirus, enteric pathogens, antigens to Giardia species, Cryptosporidia species and other pathogens, including acid-fast bacteria, enterococci, including but not limited to vancomycin-resistant enterococci (VRE), methicillin-resistant Staphylococcus aureus (MSRA), as well as any ova or parasitic bodies, or spore-forming parasites, including but not limited to Isospora, Clyslospora, and Cryptospora.

The process of fecal bacteriotherapy can include introducing a fecal sample of a healthy donor, or a donor having one or more desired characteristics, into a gastrointestinal tract of a patient to repopulate a healthy or desirable gut microbiota. In certain examples, prior to introduction of the fecal sample, the patient's intestinal flora can be disrupted using antibiotics, such that the healthy or desirable gut microbiota, once introduced into the patient, can easily populate the gastrointestinal tract.

The human fecal material is optionally filtered prior to its use in the microbiota restoration therapy.

The present disclosure is directed to methods for quantification of viable bacteria in a microbiota restoration therapy (MRT). The MRT may be used for the treatment of Clostridium difficile infections (CDI). CDI is a common nosocomial infection and is frequently associated with severe morbidity and mortality, especially in elderly patients. While CDI treatment is one example use for the MRT compositions disclosed herein, this is not intended to be limiting. Other diseases and/or conditions are contemplated. Some of the medical conditions that may be desirably impacted by treatment with MRT compositions may include cardiovascular and/or peripheral vascular disease, allergies, obesity, hypoglycemia, constipation, celiac sprue (e.g., celiac disease), gastrointestinal cancer (e.g. gastrointestinal cancer is at least one of stomach cancer, esophageal cancer, colon cancer gallbladder cancer, liver cancer, pancreatic cancer, colorectal cancer, anal cancer, and gastrointestinal stromal tumors), myoclonus dystonia, sacrolileitis, spondyloarthropatliy, spondylarthritis, proximal myotonic myopathy; an autoimmune disease nephritis syndrome, autism, travelers' diarrhea, small intestinal bacterial overgrowth, chronic pancreatitis, a pancreatic insufficiency, chronic fatigue syndrome, benign myalgic encephalomyelitis, chronic fatigue immune dysfunction syndrome, Parkinson's Disease (PD), amyotrophic lateral sclerosis (ALS), multiple sclerosis (MS), degenerative neurological diseases, Grand mal seizures or petitmal seizures, Steinert's disease, chronic infectious mononucleosis, epidemic myalgic encephalomyelitis, idiopathic thrombocytopenic purpura (ITP), an acute or chronic allergic reaction obesity, anorexia, irritable bowel syndrome (IBS or spastic colon) Crohn's disease, irritable bowel disease (IBD), colitis, ulcerative colitis or Crohn's colitis, chronic infectious mononucleosis, epidemic myalgic encephalomyelitis, acute or chronic urticarial, lupus, rheumatoid arthritis (RA) or juvenile idiopathic arthritis (JIA), pre-diabetic syndrome, fibromyalgia (FM), Type I or Type II diabetes, acute or chronic insomnia, migraines, hepatic encephalopathy, and attention deficit/hyperactivity disorder (ADHD).

In the case of humans, the present disclosure encompasses methods of treatment of chronic disorders associated with the presence of abnormal enteric microflora. Such disorders include but are not limited to those conditions in the following categories: gastro-intestinal disorders including irritable bowel syndrome or spastic colon, functional bowel disease (FBD), including constipation predominant FBD, pain predominant FBD, upper abdominal FBD, nonulcer dyspepsia (NUD), gastro-oesophageal reflux, inflammatory bowel disease including Crohn's disease, ulcerative colitis, indeterminate colitis, collagenous colitis, microscopic colitis, chronic Clostridium difficile infection, pseudemembranous colitis, mucous colitis, antibiotic associated colitis, idiopathic or simple constipation, diverticular disease, AIDS enteropathy, small bowel bacterial overgrowth, coeliac disease, polyposis coil, colonic polyps, chronic idiopathic pseudo obstructive syndrome; chronic gut infections with specific pathogens including bacteria, viruses, fungi and protozoa; viral gastrointestinal disorders, including viral gastroenteritis, Norwalk viral gastroenteritis, rotavirus gastroenteritis, AIDS related gastroenteritis; liver disorders such as primary biliary cirrhosis, hepatic encephalopathy, primary sclerosing cholangitis, fatty liver or cryptogenic cirrhosis; rheumatic disorders such as rheumatoid arthritis, non-rheumatoid arthritidies, non rheumatoid factor positive arthritis, ankylosing spondylitis, Lyme disease, and Reiter's syndrome; immune mediated disorders such as glomeruionephritis, haemolytic uraemic syndrome, juvenile diabetes mellitus, mixed cryoglobulinaemia, polyarteritis, familial Mediterranean fever, amyloidosis, scleroderma, systemic lupus erythematosus, and Behcets syndrome; autoimmune disorders including systemic lupus, idiopathic thrombocytopenic purpura, Sjogren's syndrome, haemolytic uremic syndrome or scleroderma: neurological syndromes such as chronic fatigue syndrome, migraine, multiple sclerosis, amyotrophic lateral sclerosis, myasthenia gravis, Guillain-Barre syndrome, Parkinson's disease, Alzheimer's disease, Chronic Inflammatory Demyelinating Polyneuropathy, and other degenerative disorders; sychiatric disorders including chronic depression, schizophrenia, psychotic disorders, manic depressive illness; regressive disorders including, Asbergers syndrome, Rett syndrome, attention deficit hyperactivity disorder (ADHD), and attention deficit disorder (ADD); the regressive disorder, autism; sudden infant death syndrome (SIDS), anorexia nervosa; dermatological conditions such as chronic urticaria, acne, dermatitis herpetiformis and vasculitis disorders; and cardiovascular and/or vascular disorders and diseases.

Globally, the increase in the prevalence of drug resistant organisms has created many challenges for clinicians that may pose public health risks. Infections by drug resistant organisms (e.g., vancomycin-resistant Enterococcus (VRE)) and Clostridium difficile infection share similar risk factors. VRE is a nosocomial pathogen that can be a complication among transplant and immune compromised patients. VRE carriers may also be at increased risk for infection due to VRE and also be a potential source of VRE transmissions to others. VRE shedding in stool increases with antimicrobial exposures and decreases with normalization of the intestinal microbiota after antimicrobials are discontinued. Accordingly, normalization of intestinal microbiota may not only be useful for treating Clostridium difficile infections (including chronic infections), these treatments may also be useful for treating infections by drug resistant organisms (e.g., VRE and/or other drug resistant organisms including those disclosed herein).

In some instances, the microbiota restoration therapy compositions (and/or fecal bacteriotherapy compositions) disclosed herein may be used to treat patients with infections by drug resistant organisms and/or multi-drug resistant organisms (MDRO). The drug resistant organisms may be resistant to antimicrobial agents (e.g., antibiotics, antivirals, antifungals, antiparasitics, other drugs, combinations thereof, and the like) and may include drug resistant micro-organisms such as bacteria, viruses, fungi, parasites, etc. The infections that can be treated by the microbiota restoration therapy compositions disclosed herein may be along the digestive tract or along other systems of the patient.

The microbiota restoration therapy compositions may be used to treat infections by a variety of drug resistant organisms such as vancomycin-resistant enterococci (VRE), methicillin-resistant Staphylococcus aureus (MRSA), extended-spectrum β-lactamase producing gram-negative bacteria, Klebsiella pneumoniae carbapenemase producing gram-negative bacteria, multi-drug resistant gram negative rods bacteria (e.g., such as Enterobacter species, E.coli, Klebsiella pneumoniae, Acinetobacter baumannii, and Pseudomonas aeruginosa), drug resistant Enterobacter species, multi-drug resistant tuberculosis (e.g., Mycobacterium tuberculosis), drug resistant staphylococci, drug resistant enterococci, drug resistant gonococci, drug resistant streptococci (e.g., including Streptococcus pneumoniae), drug resistant salmonella, drug resistant gram negative bacteria, drug resistant Candida, drug resistant HIV, drug resistant influenza virus, drug resistant cytomegalovirus, drug resistant herpes simplex virus, drug resistant malaria, drug resistant Plasmodium vivax, drug resistant Plasmodium falciparum, drug resistant Toxoplasma gondii, and the like, and/or other drug resistant organisms. These are just examples.

Treatment of infections by drug resistant organisms with the microbiota restoration therapy compositions disclosed herein may include treating patients with no prior history of infection with a drug resistant organism, treating patients with a single prior infection by a drug resistant organism, treating patients with two or more (e.g., two, three, four, five, six, or more) prior infections by a drug resistant organism, etc. In some instances, the microbiota restoration therapy compositions may be used to treat a patient with three prior infections by a drug resistant organism. In other instances, the microbiota restoration therapy compositions may be used to treat a patient with two prior infections by a drug resistant organism if the prior infections resulted in hospitalization, if the prior or current infections require treatment with toxic drugs, or if the prior infections were all from the same organism.

In some instances, MRT compositions can be administered to a patient using an enema or other suitable technique. However, it may be desirable to orally administer an MRT composition. In order to prepare an MRT composition in a form suitable for oral administration, a number of steps may be carried out. Generally, these steps may include collecting a fecal sample, processing the fecal sample, lyophilizing or “freeze-drying” the processed fecal sample, adding one or more additives and/or excipients, and forming an oral form of the MRT composition from the lyophilized material and additives (e.g., a tablet, capsule, liquid preparation, or the like).

Once the MRT composition has been prepared for administration to a patient, it may be desirable to quantify the viable bacterial microorganisms in the drug product. For example, the active ingredient in an MRT composition is considered to be the viable bacterial microorganisms present in the suspension and/or lyophilized product. In order to release a product based on a quality specification, the “potency” of that product must be determined (“potency” is defined in 21 CFR 600.3(s) as “the specific ability or capacity of the product, as indicated by appropriate laboratory tests or by adequately controlled clinical data obtained through the administration of the product in the manner intended to effect a given result”). It is contemplated that quantitative real-time polymerase chain reaction (qPCR) may be used to quantify the viable bacterial microorganisms, as will be discussed in more detail below.

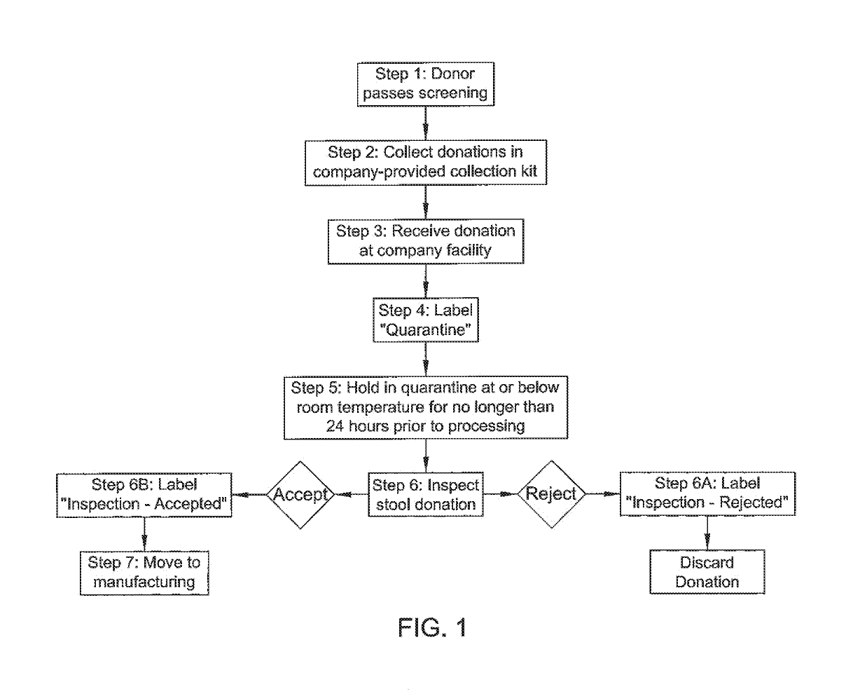

FIG. 1 is a flow chart depicting a portion of an example MRT production process. This is just an example. Other examples of screening donors, obtaining human stool samples, and processing the stool samples to a MRT product are disclosed in commonly assigned U.S. Patent Publication 2014/0363398, which is herein incorporated by reference. More particularly, FIG. 1 schematically depicts a process for collecting and inspecting a donor fecal sample. As a first step in the collecting/inspecting process, potential stool donors are screened. Once the donor passes the screening, step two may include collecting the donor's stool using a human stool collection kit as defined herein, whether at home or at a collection facility. The kit can include, but is not limited to, a clean human stool collection container with lid, a large closeable/sealable bag, a donation form and a human stool collection instruction sheet. The time and date of collection, along with donor identity and method of transport, can be recorded in order to track the time from collection to processing, and the conditions of transport. As a non-limiting example, the collection container can include an indicator of the minimum and the maximum temperature to which the sample is exposed. As another non-limiting example, one or more temperature sensitive stickers that changes color at temperatures below about 4° C. and temperatures greater than about room temperature (about 22-29° C.) can be affixed to the container.

Step three may involve transporting the sample to a processing facility. It can be appreciated that if the sample is collected at the processing facility, transporting the sample is not necessary. In some instances it may be desirable to collect the sample at the processing facility in order to more clearly establish the chain of custody of the sample. With the receipt of the first stool donation for any individual, a profile will be established for each donor. Subsequent stool samples can be subjected to a human stool test, which is utilized to match and confirm the identity of the donor with the donation. Based on prior collected samples, a human stool profile for the donor is generated and can be maintained or enhanced over repeated donations. Any new sample will be compared with this profile to confirm it is the same donor. Differentiation can be made to confirm donor identity based on the representation of Bacterioides species in the human stool. In a non-limiting example, the base set of stool samples used to create the profile is collected at the processing facility to assure donor identity in the profile samples. In another non-limiting example, the base set of stool samples used to create the profile can be collected in locations other than the processing facility, with donor identity assurance protocols appropriate to the situation or location.

Step four of the method may include labeling the donation “Quarantine” and holding the donation in quarantine at or below room temperature for no longer than in the range of 24 hours to five days prior to processing. Donations may be rejected in situations where the temperature indicator has been activated or where the time between donation and receipt exceeds 24 hours. In addition, where applicable, the human stool test results must match the donor profile. If the human stool test does not match the donor profile, the donation collected for that day will be discarded and the donor will be disqualified.

In one method of the disclosure, the human stool sample is processed within about 24 hours of collection. In another method of the application, the time of collection is recorded at the time of arrival of the stool sample at the processing facility. Step six may include inspecting the stool donation. Visual inspection can be completed upon arrival of the stool sample at the processing facility. In the event the human stool sample is loose, unformed, is not of sufficient weight (e.g., less than about 50 g), or for any other reason, including but not limited to evidence indicating poor sample quality or concerns about donor health, the sample may be rejected, labeled “Inspection—Rejected” and the donation is discarded. Further, answers to questions on the human stool collection form can be reviewed by trained personnel. Certain answers in the collection form may require ample rejection. If the sample is accepted, it may be labeled “Inspection—Accepted” and may be moved to a manufacturing process.

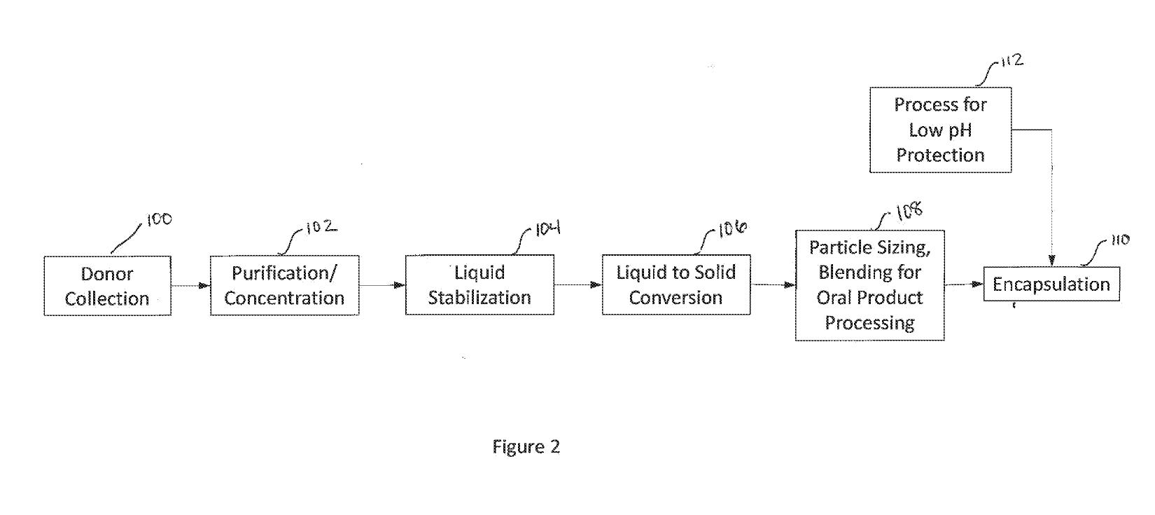

FIG. 2 is a flow chart depicting a portion of a generic illustrative method for preparing a stool sample for MRT as an oral dosage. It is contemplated that an intermediate product within the method for preparing a stool sample for MRT as an oral dosage may be suitable for MRT via an enema or gastro-nasal tube. The stool sample may first be collected and screened 100, for example, in the method described with respect to FIG. 1. Once the sample has been accepted, the sample may be purified and concentrated 102. The sample may be purified using centrifugation, membrane filtration, or a combination thereof to remove fecal material above a certain particle size. It is contemplated that since most bacteria of interest are in the range of 0.3 microns (μm) to 30 μm, the sample may be processed to remove particles greater than 50-70 μm. The sample may be processed to obtain a 75% to 90% concentration of the bacteria. This may allow for an increased flexibility in the ratio of formulation excipients to bacteria for further processing.

The sample may be membrane filtered in a number of different ways, including, but not limited to the use of filter bags, pressure filters, and/or vacuum filters. In some instances, the sample may be filtered multiple times using a smaller filter membrane with each subsequent filtering. In some instances, saline may be added as a diluent in a ratio of 1:3 (stool to saline), although this is not required. In other instances a mixture of saline and a cryoprotectant (e.g., polyethylene glycol (PEG) 3350) may be used as a diluent. The PEG concentration of the diluent can be approximately about 30-90 g/liter (or about 10-90 g/liter). The PEG concentration of the diluent can also be approximately between about 25-75 g/liter. In one example, the ratio of saline/PEG mixture to stool sample is 2:1, or 2 mL saline/PEG mixture to 1 gram human stool. As a non-limiting example, approximately 100 mL of saline/PEG mixture can be used for 50 g of human stool. While saline/PEG may be suitable for use as a diluent (and/or cryoprotectant), this is not intended to be limiting. Other cryoprotectants may also be utilized. For example, dextrose, betaine, glycine, sucrose, polyvinyl alcohol, Pluronic F-127, mannitol, tween 80, ethylene glycol, 1,3-propanediol, hydroxypropyl cellulose, glycerol, PEG/glycerol mix, propylene glycol, or combinations thereof may be used as cryoprotectants. These materials may be used alone or in combination with a solvent such as saline.

In one example, the sample may be placed in a 500 μm filter bag, with or without a diluent, and agitated using, for example, Stomacher agitation at 230 rpm for approximately 2 minutes to obtain a filtrate having a particle size of approximately 500 μm or less. This filtrate may then be placed in a filter bag having a pore size smaller than 500 μm, for example, 280 μm. The sample may be agitated again using, for example, Stomacher agitation at 230 rpm with or without a diluent for approximately 4 minutes to obtain a filtrate having a particle size of approximately 280 μm or less. This filtrate may be placed in another filter bag having a pore size smaller than, for example, 280 μm, such as, but not limited to 60 μm. The sample may be agitated again using, for example, Stomacher agitation at 230 rpm with or without a diluent for approximately 4 minutes to produce a filtrate having a particle size of approximately 50-70 μm or less.

In another example, the sample may be placed in a 500 μm filter bag, with or without a diluent, and agitated using, for example, Stomacher agitation to obtain a filtrate having a particle size of approximately 500 μm or less. This filtrate may then be processed using a pressure filter having a pore size of approximately 160 μm and the resulting filtrate processed using a pressure filter having a pore size of approximately 60 μm. In some instances, the sample may be need to be processed a second time using a bag filter having a pores size between 160 μm and 500 μm prior to using the pressure filter.

In another example, the sample may be placed in a 500 μm filter bag, with or without a diluent, and agitated using, for example, Stomacher agitation to obtain a filtrate having a particle size of approximately 500 μm or less. This filtrate may then be processed using a vacuum filter having a pore size of approximately 160 μm and the resulting filtrate processed using a vacuum filter having a pore size of approximately 60 μm. In some instances, the sample may be need to be processed a second time using a bag filter having a pores size between 160 μm and 500 μm prior to using the vacuum filter.

Once the sample has been processed to have a particle size of approximately 60 μm or less, the sample may then be washed and further concentrated using a centrifuge. In some instances, centrifuge tubes may have a volume in the range of 50 to 500 mL, or more. The filtered suspension is filled to approximately 20 to 80% of the volume of the centrifuge tube. In one example, the samples may be centrifuged at 1100 to 3600 revolutions per minute (rpm) for 10 to 15 minutes cycles. In another example, the samples may be centrifuged at a rate such that the centrifugal force is in the range of about 8-12,000 g (e.g., about 10,000 g) for 15-45 minutes or 20-30 minutes. The centrifuge may be ramped up or gradually accelerated to the speed needed to create a centrifugal force in the range of about 8-12,000 g (e.g., about 10,000 g). It is further contemplated that the centrifuge may also be slowly ramped down or decelerated when the centrifugation process is complete. In some instances, it may be desirable to decelerate the centrifuge as slowly as possible so that the return to atmospheric pressure is slow so as to protect the bacterial cells from potentially bursting. The supernatant is removed and the remaining material in the tube is the purified intermediate MRT composition. This may result in a product that has been concentrated by approximately 60%. In some instances, the centrifugation process may be a 2-tiered process. For example, the product may first undergo a “pre-spin” (for example 300 g for 2-5 minutes) to remove fecal fibrous material and then may undergo a longer centrifugation to concentrate the product. It is further contemplated that volumes of up to 300 mL may be centrifuged without resulting in a drop in the amount of concentration. The resulting MRT composition is a bacterial suspension having a particle size of 70 μm or less and a bacterial concentration on the order of approximately 1×10¹⁰ CFU/g. The resulting MRT composition may also be stable for 3 weeks at refrigeration conditions. This resulting MRT composition may be suitable for delivery to a patient via an enema or gastro-nasal tube. Further processing may be required to convert the liquid MRT composition to a solid suitable for oral delivery.

In some embodiments, centrifugation alone can be used multiple times for purification and concentration. However, the particle size of the bacterial suspension may still be in a range (e.g. greater than 60 μm) that clogs pipet tips. However, in some instances, wide pipette tips may be used. Whether this is successful or not is dependent on the input fecal material, which is variable. It is further contemplated that a system of separators and decanters could be used if the batch size was in the range of several tens of liters, or more. However, this may not be required if the starting product has been previously processed.

In some embodiments, it may be desirable to stabilize the processed sample in suspension 104 at refrigeration conditions for a period of time in the range of one to two weeks. In some instances, removal of the fecal material and replacement with carriers or excipients which are soluble in an aqueous solution may allow the bacteria to be suspended in the liquid and further processed without stability concerns. Considerations for these excipient solutions may be pH, concentration, and isotonicity or isosmolality. Excipients may be selected based on protein and monoclonal antibody formulations and their proposed role in stabilizing biologics. Some example excipients that may be used to provide liquid stabilization 104 of the sample may include, but are not limited to: salt (NaCl), sucrose, trehalose, L-arginine monohydrochloride, and/or PEG 3350.

It is contemplated that similar excipients may also be used to protect the bacteria during membrane filtration. For example, Farber and Sharpe in Applied and Environmental Microbiology, August 1984, P. 441-443 state that bacterial recovery is improved in the presence of certain food debris (carrots, cheese, peaches, tuna)—pH may be important—pH 5.88 to 6.40 for carrots, pH 4.75-5.02 for cheese, pH 5.9 to 6.2 for tuna, pH 3.3 to 4.05 for peaches. The presence of sugars, carbohydrates, or proteins may be important, properties of these foods that coat the bacteria, support bacterial growth (pre-biotic activity) or support the bacterial cell wall during filtration may be important.

From the time of producing the standardized product through the time of administration to the patient, the standardized product must be maintained viable. This can include using a frozen storage technique and cryoprotectant to maintain viability. For example, polyethylene glycol (PEG) can be used as an effective cryoprotectant for MRT products. Time of storage, thawing technique, shipping technique and handling of the thawed product are also factors that affect viability and are defined herein. In one embodiment, the cryoprotectant polyethylene glycol (PEG) can be mixed with the human stool sample and isotonic saline at the time of processing. PEG can be added at a concentration from about 0.1 g/ml. to about 70 g/ml, or from about 2 g/ml to about 68 g/ml, or from about 4 g/ml to about 65 g/ml, or from about 5 g/ml to about 60 g/ml. The PEG used can have an average molecular weight of about 600 to about 20000. In some embodiments, the PEG has an average molecular weight of about 2000 to about 4000, for example about 3350 as provided in the for mulation of PEG 3350. Other cryoprotectants may be used such as dextrose, betaine, glycine, sucrose, polyvinyl alcohol, Pluronic F-127, mannitol, tween 80, ethylene glycol, 1,3-propanediol, hydroxypropyl cellulose, glycerol, PEG.

Suitable carriers may vary with the desired form and mode of administration of the composition. For example, they may include diluents or excipients such as fillers, binders, wetting agents, disintegrators, surface-active agents, glidants, lubricants, and the like. Typically, the carrier may be a solid (including powder), liquid, or combinations thereof. Each carrier is preferably “acceptable” in the sense of being compatible with the other ingredients in the composition and not injurious to the subject. The carrier may be biologically acceptable and inert (e.g., it permits the composition to maintain viability of the biological material until delivered to the appropriate site).

Oral compositions may include an inert diluent or an edible carrier. For the purpose of oral therapeutic administration, the active compound can be incorporated with excipients and used in the form of tablets, troches, or capsules, e.g., gelatin capsules. Oral compositions can also be prepared by combining a composition of the present disclosure with a food. In one embodiment a food used for administration is chilled, for instance, ice cream. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, orange flavoring, or other suitable flavorings. These are for purposes of example only and are not intended to be limiting.

Once the sample has been purified and stabilized in an aqueous suspension (e.g. the above described MRT composition), which at this point may be suitable for delivery via a gastro-nasal tube or an enema, the sample may be further processed to be suitable for an oral delivery, such as in the form of tablets, troches, or capsules. For example, the aqueous solution may be converted to a solid 106. A list of bacterial processing techniques can be found in Martin et al., Innovative Food Science and Emerging Technologies, 27 (2015) 15-25.

In some instances, lyophilization, or freeze-drying, may be used to convert the sample from a liquid to a solid. The sample may be provided with a cryoprotectant such as, but not limited to PEG, skim milk, charcoal, ascorbic acid or a combination thereof to protect the bacteria from the effects of freezing. The sample may also be provided with a lyoprotectant such as, but not limited to sucrose, inositol, trehalose, glycerol, or a combination thereof. In some instances, the sample may also be provided with an enrichment material which may provide acid buffering. Alternatively or additionally, the enrichment material may also keep the bacteria more active which may facilitate analytical testing. Some example enrichment materials may include, but are not limited to skim milk, charcoal, gelatin, ascorbic acid, GI media, or combinations thereof. Alternatively or additionally, an oxygen scavenger may be added to the sample prior to and/or after lyophilization. While not wishing to be bound by theory, it is believed that an oxygen scavenger may improve the stability and/or viability of the sample. It is contemplated that lyophilization tubes may include an insert that can be used to expel a lyophilized pellet from the lyophilization tube after freeze-drying. The width of the lyophilization tube may be smaller than the width of a capsule shell for oral treatment. This may allow for the displacement of a tray of pellets directly into the capsule shells. It is contemplated that this may reduce or eliminate the need for particle sizing of the formulation or blending it further 108 for improvement in flow properties into the capsule. The dose may also be determined by pellet size. In some instances, a pellet produced in the lyophilization process may include approximately 4.5×10⁸ colony forming units (CFU) (CDC). A size 0 capsule may accommodate three pellets. Thus, a capsule may include approximately 6.7×10⁹ CFU (CDC). Eight capsules taken twice a day may be required to be equivalent to one enema dose. Further, there may be no need to test for homogeneity of the batch of pellets that are mixed together prior to capsule filling. In some instances, tampering may allow for a greater concentration or number of pellets within each capsule. For example, tampering of the pellets within the capsule may allow for about 2-4 times (e.g., about 2.5 times) the number of pellets in each capsule (e.g., without tampering each capsule may accommodate 2-4 or about 3 pellets whereas with tampering each capsule may accommodate about 7-10 or about 8 pellets). This may help to reduce the number of capsules a patient may need to take in order to achieve the desired dose.

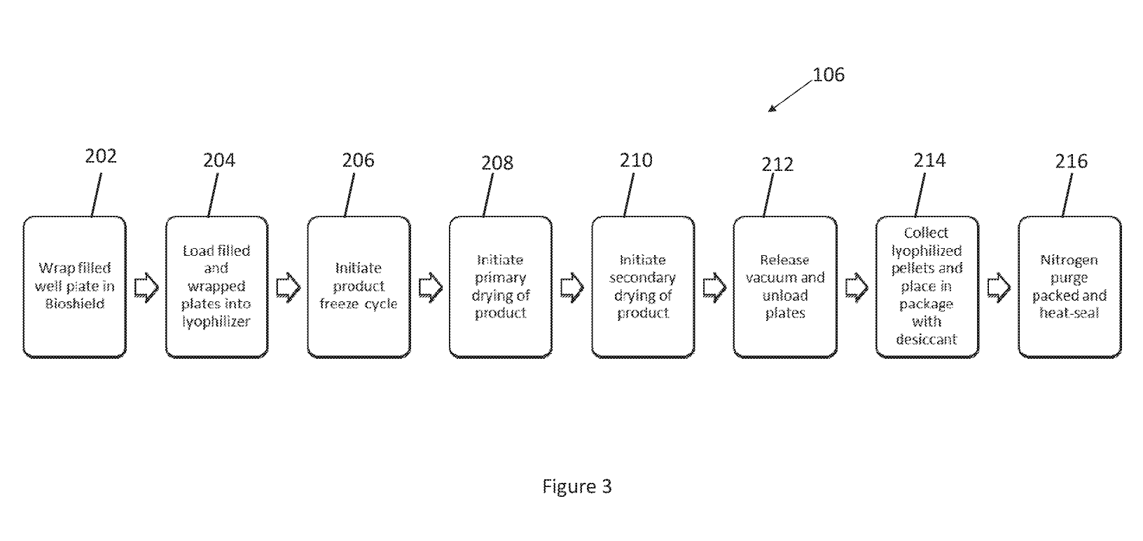

An illustrative lyophilization procedure (e.g. solid to liquid conversion) 106 will be described with respect to FIG. 3. The MRT composition, or purified intermediate, may be mixed at a 1:1 ratio with a lyophilization excipient solution. The lyophilization excipient solution may be comprised of 2.3% PEG 3350, 1% glycerin, 10% trehalose, and 10% sucrose. However, other lyophilization excipients may be used. Prior to adding the excipient solution to the purified intermediate, the lyophilization excipient solution (without glycerin) is filtered through a 0.2 μm filter. The glycerin is autoclaved at 121° C. for a minimum of 15 minutes and added aseptically. Once the lyophilization excipients and purified intermediate have been mixed (lyophilization suspension), a single two hundred microliter (200 μL) aliquot of the lyophilization suspension is placed in each well of a 96-well plate and lyophilized. To perform the lyophilization, once filled, the 96-well plate may be wrapped in sterile bioshield, as shown at step 202. Other plate sizes are also contemplated. After all plates are wrapped, they may be immediately transported and loaded into the lyophilizer, as shown at step 204. The lyophilizer may be sealed and the lyophilization cycle initiated. Product is frozen by lowering the product shelf temperature to a range of approximately-40° C. to −45° C., as shown at step 206. After the product is frozen, primary drying (sublimation) occurs by applying vacuum and elevating the shelf temperature up to 0° C., as shown at step 208. A secondary drying step is initiated to further reduce water content and bring the product to ambient temperature (approximately 25° C.), as shown at step 210. The vacuum is released at the end of the secondary drying step and the product is removed from the lyophilizer, as shown at step 212. Product may be placed inside an anaerobic chamber for collection of the lyophilized aliquots. The lyophilized aliquots may be in pellet form and are transferred to a packaging with desiccant, as shown at step 214. Filled packages may be purged with nitrogen gas and heat-sealed, as shown at step 216.

In some instances, it may be desirable for the lyophilized pellets to have a glass transition temperature (T_{g}) of greater than 30° C. This may result in a final product that is stable at room temperature. The glass transtion temperature may as also be used a tool for screenting the product received form the lyophilization process and/or for verifying the stablility of the final product.

In other instances, it may be desirable to preserve the sample through vaporization foam drying. It is contemplated that traditional excipients and equipment may be used with this process. Higher excipient concentrations and optimal process parameters to produce foam during processing may result in low water content formulations. The lower the water content; the greater the probability of stability at room temperature. Referring again to FIG. 2, once the sample has been dried 106, the sample may be further processed to achieve a desired particle size and/or blending 108 in order to prepare the sample for oral product processing.

In yet other embodiments the liquid sample may be optionally microencapsulated by lipids to protect from bile, alginates, and/or polymers. Once the sample has been microencapsulated, the sample may be further processed to achieve a desired particle size and/or blending 108 in order to prepare the sample for oral product processing. After the sample has been processed to a desired particle size and/or blended 108 in order to prepare the sample for oral product processing, the sample may be encapsulated 110, as described in more detail below. It is contemplated that the encapsulation process may provide for low pH protection 112. For example, the encapsulation process may prevent or substantially prevent capsule shells, tablets, and/or troches from breaking down in the acidic environment of the stomach such that the MRT composition is released in the desired portion of the intestinal tract. It is contemplated that an enteric coated capsule may be needed to provide for protection in the stomach and have disintegration of the capsule in the small and large intestine. In some instances, the capsules may be pan coated with the enteric coating. Enteric coating materials may include fatty acids, waxes, shellac, plastics, and plant fibers. Pan coating of hydroxypropyl methylcellulose (HPMC), or also called Hypromellose capsules, will protect at low pH and also help to protect from moisture.

Some suitable capsules may include DRcaps™ and Vcaps™ available from Capsugel®. Likewise, AR caps having a composition of 60% HPMC and 40% HPMCP (hypromellose phthalate) may have the same properties. Capsule types that are not gelatin may contain less water (gelatin caps usually 10 to 12% water, versus other polymer capsules have 3-4% or less water). Banding of the capsule with polymers that are insoluble in low pH environments may be required, as will be discussed in more detail below. In other instances, the capsules may be stacked such that 2 or more capsules are used to enclose the sample. For example, the sample may be placed in a capsule and then that capsule placed in another larger capsule.

Upon receipt of the lyophilized intermediate, it may be removed from the packaging and filled into capsules. The lyophilized intermediate may also be sampled and the total viability is measured via a PMA-qPCR method. Encapsulation may be conducted in a nitrogen-purged area at ambient temperature to minimize the exposure of the lyophilized intermediate to oxygen. The lyophilization intermediates are encapsulated in a hypromellose capsule. Multiple lyophilized intermediates can be loaded into a hypromellose capsule depending on the capsule size (e.g., sizes 1, 0, or 00).

The capsule may then be banded. In some instances, the capsules may be banded with hypromellose. In other instances, the banding material may be an anionic copolymer based on methacrylic acid and methyl methacrylate, such as, but not limited to Eudragit® L100. In yet other instances, the banding material may be hypromellose phthalate or hypromellose acetate succinate. These are just examples. The banding material may be any material which is resistant to low pH environments (e.g. the stomach) and degrades in high pH environments (e.g. the intestinal tract). A consistent banding thickness is applied to each capsule so the disintegration performance meets the acceptance limit. Capsules are stored at refrigeration conditions, 5±3° C. in a nitrogen-purged bulk plastic container or packaged with desiccant. Encapsulated and banded drug product may be packaged with desiccant and heat-sealed. In some instances, the encapsulated and banded drug product may be packaged in individual dosage quantities in metallized polyester/polyethylene bonded film. This may minimize the exposure of the drug product to oxygen and/or moisture which may cause degradation of the product. The metallized polyester/polyethylene bonded film may have a moisture vapor transmission rate of 0.02 gr/100 in² and an oxygen transmission rate of 0.0402 /mL/100 in² in 24 hours. The bonded film packets may be provided to the patient in a child-resistant container to meet the need for child-resistant clinical supply packaging. The child-resistant container may be a 40 dram (2.5 ounces) green pharmacy vial with a child-resistant cap. The vial may be made of translucent, light resistant polypropylene. The low density polyethylene (LDPE) child-resistant cap helps prevent unauthorized access by requiring that the user push down and rotate the cap to open the container.

As described above, it may be desirable to quantify the viable bacterial microorganisms in a drug product for delivery via a gastro-nasal tube, an enema and/or a capsule or tablet. A molecular-based approach, such as PMA (propidium monazide)-qPCR (quantitative polymerase chain reaction) assays may be useful for quantification of viable bacteria. Quantitative PCR alone may not be sufficient to determine the number of viable bacteria in a sample because DNA from both live and dead cells is amplified. By utilizing PMA treatment in combination with qPCR, the number of viable bacterial cells in the sample can be determined. Briefly, the PMA-qPCR assay is a two part process. First, samples are treated with a photo-reactive DNA-binding dye called propidium monoazide (PMA). Upon photo activation, PMA intercalates between the bases of DNA and renders it unable to be PCR amplified. Due to the chemical structure of PMA, it cannot penetrate bacterial cell membranes. Therefore, the DNA in viable cells (intact cell membrane) is protected from PMA, while the DNA from dead cells (ruptured cell membrane) is bound by PMA and unable to be PCR amplified (Fittipaldi M., Nocker A., Codony F. 2012. Progress in understanding preferential detection of live cells using viability dyes in combination with DNA amplification. J Microbiol Methods. 91 (2): 276-89.). The second portion of this method utilizes quantitative PCR (qPCR). Quantitative PCR is a culture-independent assay that allows the number of organisms in a sample to be determined based on the gene copy number that is detected. Primers may be designed or chosen to amplify a particular region of a particular gene.

It is contemplated that for analyzing a microbiota restoration therapy product, primers may be chosen and/or designed to amplify the V3 region of the 16 s rRNA gene. This region was chosen because it is a hypervariable region flanked by highly conserved sequences and is the proper sequence length for PMA-qPCR. In some instances, the primer may be a custom-designed JE 341F (forward sense primer, listed in Table 1 below and identified in the Sequence Listing as SEQ ID NO. 1) and/or V3-R1 (standard reverse antisense primer, listed in Table 1 below and identified in the Sequence Listing as SEQ ID NO. 2) may be used, as shown in Table 1 below. These are just examples.

TABLE 1
Primer sequences

| Primer | Sequence 5′-3′ |
|---|---|
| JE 341F | CMTACGGGNBGCASCAG |
| | SEQ ID NO. 1 |
| V3-R1 | GACTACNVGGGTATCTAATCC |
| | SEQ ID NO. 2 |

Other primers may be chosen and/or designed to amplify the V3 region of the 16 s rRNA gene. It is further contemplated that other primers may be chosen and/or designed to amplify other regions and/or genes, as desired. These primers (JE 341F and/or V3-R1) may have a 95.55% coverage rate of all prokaryotes (Ribosomal Project Probe Match Program (RDP)). This means they are capable of amplifying the V3 region of the 16 s rRNA gene from 95.55% of all prokaryotes. Therefore, the ‘total’ number, or 100% of the bacteria in the sample may not be detected in every sample processed.

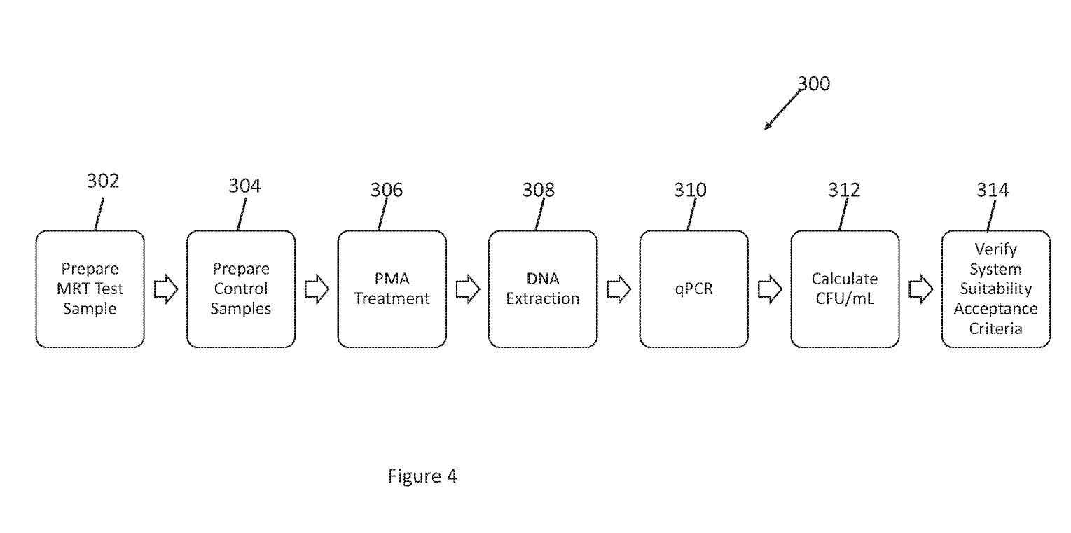

FIG. 4 is a flow chart depicting an illustrative method 300 for determining the number of viable microorganisms in an MRT drug substance using PMA™-qPCR. It is contemplated that the method may be the same for an MRT drug substance prepared for delivery via an enema tube or via an enema or gastro-nasal tube. The MRT drug substance may be obtained and prepared 302. In some instances, a minimum of 200 microliters (μL) of the MRT drug substance may be required to run an analysis. It is contemplated that the MRT drug substance may be stored at −80 (−10/+20)° C. prior to preforming the analysis. However, refrigeration may not be necessary for all samples. To prepare the MRT drug substance for testing, the MRT drug substance may be diluted by a factor of 100. For example, 9.9 milliliters (mL) of saline (0.9%) may be added to 100 μL of MRT drug substance. The diluted MRT drug substance, which may be mixed in the same way as the positive control, may be placed into a centrifuge tube. In some instances, approximately 500 μL of diluted MRT drug substance (hereinafter referred to as the “test sample”) may be placed into a 1.5 mL micro-centrifuge tube. One or multiple tests may be executed using the MRT drug substance where additional volume is available. Single or multiple MRT drug substances may be prepared in unique, separate micro-centrifuge tubes per process.

Control samples may also be prepared 304. It is contemplated that both positive controls and negative controls may be used for comparison purposes. Positive controls may consist of a product reference standard (PRS) which may include an MRT drug substance maintained for quality testing. In some instances, the positive control samples and negative control samples may be stored at −80 (−10/+20)° C. The control samples may be removed from a freezer and thawed at room temperature for approximately 15-30 minutes, although other time periods of less than 15 minutes or greater than 30 minutes are contemplated. The positive control sample may be diluted by a factor of five with saline (0.9%). The negative control may not be diluted with saline, although dilution factors below or above five in saline may be used. For example, 400 μL of saline may be added to 100 μL of the positive control. In some embodiments, the saline may be placed in a 1.5 mL micro-centrifuge tube and the positive control added to the micro-centrifuge tube. This is just an example. It is contemplated that the positive control may be placed in the 1.5 mL micro-centrifuge tube and the saline then added to the micro-centrifuge tube. The micro-centrifuge tube may be swirled or vortexed (either by hand or machine) briefly to mix the saline and positive control.

Once the test samples and control samples have been mixed, the test samples and control samples may be treated with PMA, as shown at 306. 1.25 μL of a 20 millimolar (mM) PMA stock solution may be added to the 500 μL aliquots of test samples, positive control samples, and negative control samples. To prepare a 20 mM stock solution of PMA dye, 1 milligram (mg) of PMA dye may be dissolved in 98 μL of sterile solution. The PMA stock solution may be sable at −20° C. for approximately 6 months. It may be desirable to store the PMA stock solution in a UV resistant container or otherwise protect the PMA stock solution from light. The PMA stock solution may be briefly centrifuged prior to use to collect the solution at the bottom of the vial, although this is not required.

Each of the micro-centrifuge tubes may be inverted five times to mix the PMA stock solution with the samples. This is just an example. The micro-centrifuge tubes may be inverted any number of times desired or mixed in another manner, such as, but not limited to swirling. During the mixing procedure, it may be desirable to minimize light exposure to the PMA dye. The test samples and control samples may be incubated in the dark for approximately 6 minutes±1 minute. The test samples and control samples may each be inverted two times at approximately 2 minutes into the incubation period and 4 minutes into the incubation period.

After the incubation period is over, 100 μL from each test and control sample may be added into three individual wells for a total of 300 μL per sample within a lidded V-bottom 96-well plate which may be uniquely labeled to maintain sample traceability. Multiple samples may be included per 96-well plates per individual, unique wells. It is contemplated that differently shaped plates and/or differently sized (e.g. fewer than or greater than 96 wells) may also be used. The lid may then be removed from the plate and the plate placed under a light emitting diode (LED) light for a total of 10 minutes±1 minute. In some instances, the LED light may be a 1720-lumen LED work light. However, other LED lights may be used. The top of the plate may be placed approximately 2.5 centimeters (cm)±0.5 cm from the surface of the light source. After approximately 5 minutes of light exposure±0.5 minutes, the plate may be removed from under the light source. The samples may be mixed by pipetting up and down five times with a multichannel pipette. It is contemplated that the samples may be mixed using other methods or by pipetting up and down any number of times (e.g. fewer than or greater than five). It may be desirable to set the volume of the pipette to approximately 70 μL to reduce or eliminate bubbling. The plate may be placed back under the light and exposed to the light for another 5 minutes±0.5 minutes.

After the test samples and controls samples have been exposed to the LED light, the DNA extraction may begin, as shown at 308. The test and control samples may be centrifuged in the 96-well plate at 2,100 g for approximately 5 minutes±1 minute. The rpms required to achieve the desired acceleration may be dependent upon the radius of the centrifuge. After centrifugation, the supernatant may be carefully removed with a multichannel pipette to avoid disturbing the pellet at the bottom of the centrifuge tube. Each of the pellets may then be re-suspended in 100 μL of phosphate-buffered saline (PBS) buffer. Each of the pellets may be mixed with the PBS buffer may be mixed by pipetting up and down five times with a multichannel pipette. It is contemplated that the samples may be mixed using other methods or by pipetting up and down any number of times (e.g. fewer than or greater than five). The re-suspended test and control samples may be centrifuged at 2,100 g for approximately 5 minutes±1 minute. After centrifugation, the supernatant may be carefully removed with a multichannel pipette to avoid disturbing the pellet at the bottom of the centrifuge tube. Each of the pellets may then be re-suspended in 50 μL PrepMan® Ultra Sample Preparation Reagent, available from Thermo Fisher Scientific. Each of the pellets may be mixed with the PrepMan® Ultra Sample Preparation Reagent may be mixed by pipetting up and down twelve times with a multichannel pipette. It is contemplated that the samples may be mixed using other methods or by pipetting up and down any number of times (e.g. fewer than or greater than twelve). The test samples and control samples (mixed with PrepMan® Ultra Sample Preparation Reagent) may then be transferred from a V-bottom 96-well plate to a secondary 96-well high profile semi skirted PCR plate.

The plate, including the test samples and the control samples, may be placed into a freezer at −80° C. for approximately 5 to 30 minutes. The plate may be removed and placed into a thermal cycler (also known as a thermocycler, a PCR machine, or a DNA amplifier). The thermal cycler may be programmed to run a program or protocol which heats the samples to 95° C. for 3 minutes±0.5 minutes and then cooled to 4° C. for 30 seconds±10 seconds. Following the heat cycling, the samples may be transferred to a V-bottom 96-well plate and centrifuged at 2,100 g for approximately 5 minutes±1 minute. After centrifugation, approximately 30 μL of the supernatant may be carefully removed with a multichannel pipette to avoid disturbing the pellet at the bottom of the plate. The 30 μL of supernatant may then be placed into a clean 96-well plate. These 30 μL aliquots are the DNA samples (both test samples and control samples) that will be analyzed. It is contemplated that at this point, the supernatant may be stored in a freezer at approximately −20° C. The DNA samples may then be diluted by a factor of 50 in ultra-pure water. If the DNA samples were previously frozen, they should be thawed before dilution. In one embodiment, 196 μL of molecular grade water may be placed into each well of a sterile 96-well plate. A V-bottom PCR plate or similar may be used to contain samples. 4 μL from each of the 30 μL DNA samples may be added to the 196 μL of molecular grade water for a total of 200 μL in each well.

After dilution, the DNA samples may be prepared for the qPCR reaction, as shown at 310. A qPCR standard, such as G-Block dehydrated DNA that has been rehydrated in a Tris EDTA (TE) buffer at a 1:10 dilution or equivalent, may be prepared and run with the MRT test samples (e.g. unknowns) to provide a standard curve. The standard curve may allow for the gene copy number for each unknown sample to be determined. It is contemplated that standard dilutions may be 10⁻⁵, 10⁻⁶, 10⁻⁷, 10⁻⁸, and 10⁻⁹.

A qPCR reaction mixture may be provided in each individual well/tube of the PCR plate in addition to the template (e.g. standard dilutions, positive control, negative control, or MRT test sample). It should be noted that only a single template is placed in each individual well. The qPCR reaction mixture may include the reagents and volumes (per tube) listed in Table 2 below.

TABLE 2
qPCR Reaction Master Mixture (per well)

| Reagent | Volume (μL) |
|---|---|
| SsoAdvanced ™ Universal Inhibitor- | 10 |
| Tolerant SYBR ® Green Supermix | |
| (available from Bio-Rad) | |
| Primer 1 (JE 341F) | 0.4 |
| Primer 2 (V3-R1) | 0.4 |
| Molecular Grade Water | 4.2 |

To prepare the qPCR master mix, multiply the number of qPCR wells needed for the qPCR run by each of the volumes listed in Table 2, and add those volumes to the 1.5 mL micro-centrifuge tube. In some instances, it may be desirable to add extra of each reagent, for example approximately 5% extra, to allow for possible transfer losses. For example, if 50 wells are to be used, 500 μL of SsoAdvanced™ Universal Inhibitor-Tolerant SYBR® Green Supermix is required (e.g. 50 wells times 10 μL per well). To allow for possible transfer losses 25 μL (e.g. approximately 5%) may be added for a total of 525 μL. It is contemplated that less than 5% or greater than 5% may be added to the required volumes to allow for possible transfer losses, as desired. The reagents shown in Table 2 may be added in the following order to the centrifuge tube: Molecular grade H20, primer 1, primer 2, SsoAdvanced™ Universal Inhibitor-Tolerant SYBR® Green Supermix. Vortex briefly to mix. It is contemplated that the centrifuge tube may be mixed by hand or machine, as desired.

15 μL of the qPCR master mix may be added to each well of a 96-well qPCR plate that will be used for the qPCR procedure. It is contemplated that any number of wells may be used depending on the number of MRT test samples, control samples, and/or standard dilutions to be tested. For example, all 96 wells or fewer than 96 wells may be used. The qPCR master mix should be added only to number of wells that will include a test sample, control sample, and/or standard dilution. In some instances, at least 3 wells may include no template control (NTC) samples. These samples may not include any colony forming units (CFU). 5 μL of molecular grade water may be added to the sample wells designated as NTC. After the addition of the molecular grade water, the NTC sample wells may be covered to prevent or minimize contamination. 5 μL of the standard dilution series, positive control, negative control, and/or test samples may be added to the designated wells. In some instances, a template may be provided to illustrate where test samples (unknowns), positive controls, negative controls, NTC's, and standards should be placed in the qPCR plate. After the templates have been added to the qPCR master mix, the plate may be sealed. In some instances, the seal may be a Bio-Rad Microseal® ‘B’ Seal, although this is not required. The qPCR plate may then be centrifuged for approximately one minute at approximately 2100 RPM to ensure that each of the qPCR reaction mixtures are at the bottom of the tube. The RPMs may vary depending on the size of the centrifuge used.

Following centrifugation, the qPCR plate may be placed in the qPCR detection system. In some instances, the detection system may be a BIO-RAD CFX96 Touch™ Deep Well Real-Time PCR Detection System, although other real time detection systems may also be used. The qPCR detection system may be programmed to run the following thermocycle protocol outlined in Table 3 below.

TABLE 3
Thermocycle Parameter

| Step | Temperature (° C.) | Time | |
|---|---|---|---|
| 1. Initial Denaturation | 98 | 3 | minutes |
| 2. Denaturation | 95 | 10 | seconds |
| 3. Annealing | 56 | 30 | seconds |

Steps 2 and 3 may be repeated 39 times for a total of 40 denaturation/annealing cycles. During the annealing step, the fluorescence or expression intensity of adherence of the PMA dye to exposed bacterial DNA may be recorded. Once the qPCR is complete, the total CFU/mL for each MRT test sample can be determined, as shown at 312, using the following equation:

$$\text{Test Sample} \frac{CFU}{mL} = \frac{( Test Sample - Sample Background ) * \text{Dilution Factor}}{\text{Average 16s gene copy number per} \text{CFU}}$$

where Sample Background, which may be established using a known ratio of live/dead organisms such as E. coli or product reference standard, is the average background gene copy number before CFU transformation, Test Sample is the gene copy value output by the detection system based on the standard curve, the Dilution Factor is 5.0×10⁵, and the Average 16 s gene copy number per CFU is 5. Sample CFU/mL may be calculated by a computer program or manually by the operator. It is contemplated that multiple test samples may be taken from each donation and/or MRT product. The CFU/mL for each of these samples may be averaged to determine an average CFU/mL for the donation and/or MRT product.

In order to ensure accurate results, it may be desirable to verify certain system suitability acceptance criteria, as shown at 314. For example, the following criteria may need to be met or the results may be invalided:
The ratio of the average positive control raw gene copy number to average negative control raw gene copy number must be >1500:1.
The average negative control quantification cycle (C_{q}) must be within +/−5 C_{q} of the average NTC C_{q} value.
The positive control CFU/mL must be +/−1 log of known CFU/mL concentration as determined by plating on agar media
The qPCR standard curve must have an r² value>0.97.
The qPCR standard curve must have an E value within the range of 100±15%.
The average NTC C_{q} value must be >31.00. The average C_{q} for NTC's must be >2 C_{q} values from any average unknown C_{q} value in order to support quantification of the unknown.
   In some instances, the qPCR run may be repeated while in other instances, the entire method 300 may need to be repeated.

It should be understood that this disclosure is, in many respects, only illustrative. Changes may be made in details, particularly in matters of shape, size, and arrangement of steps without exceeding the scope of the disclosure. The invention's scope is, of course, defined in the language in which the appended claims are expressed.

What is claimed is:

1. A process for quantifying viable bacterial microorganisms in a microbiota restoration therapy (MRT) drug substance, the process comprising:
preparing an MRT drug substance test sample from a MRT drug substance;
preparing a control test sample from a control sample;
treating the MRT drug substance test sample and the control test sample with propidium monazide (PMA);
aliquoting a portion of the MRT drug substance test sample into a plurality of individual wells of a test plate;
aliquoting a portion of the control test sample into a plurality of individual wells of the test plate;
exposing the test plate to a light for in the range of 10 minutes extracting DNA samples from the MRT drug substance test sample in each of the plurality of individual wells and the control test sample in each of the plurality of individual wells;
diluting the DNA samples from the MRT drug substance test sample in each of the plurality of individual wells and the control test sample in each of the plurality of individual wells with molecular grade water;
adding a quantitative polymerase chain reaction (qPCR) mixture to a plurality of wells of a second test plate;
adding the diluted DNA samples from the MRT drug substance test sample in each of the plurality of individual wells and the control test sample in each of the plurality of individual wells of the test plate to the plurality of wells of the second test plate centrifuging the second test plate;
placing the second test plate in a qPCR detection system and initiating a thermocycler protocol; and
calculating the total colony forming units (CFU) per milliliter (mL) (CFU/mL) of each test sample in the plurality of individual wells of the second test plate.

2. The process of claim 1, wherein preparing the MRT drug substance comprises adding about 9.9 milliliter of 0.9% saline to 100 microliters of the MRT substance product.

3. The process of claim 1, wherein the control sample comprises a positive control sample.

4. The process of claim 3, wherein the positive control sample comprises a product reference standard including an MRT drug substance.

5. The process of claim 1, wherein the control sample comprises a negative control sample.

6. The process of claim 1, wherein the control sample comprises more than one control sample.

7. The process of claim 6, wherein the more than one control sample includes a positive control sample and a negative control sample.

8. The process of claim 1, wherein treating the MRT drug substance test sample and the control test sample with PMA comprises adding 1.25 microliters (μL) of a 20 millimolar of PMA stock solution to 500 μL aliquots of the MRT drug substance test sample and the control test sample.

9. The process of claim 8, wherein treating the MRT drug substance test sample and the control test sample with propidium monazide (PMA) further comprises mechanically mixing the MRT drug substance test sample and the control test sample with the PMA.

10. The process of claim 8, wherein treating the MRT drug substance test sample and the control test sample with propidium monazide (PMA) further comprises incubating the MRT drug substance test sample and the control test sample in a dark environment after mixing the MRT drug substance test sample and the control test sample with the PMA.

11. The process of claim 1, wherein during the step of exposing the test plate to a light for in the range of 10 minutes each MRT drug substance test sample and each control test sample in the plurality of individual wells are mixed at least one time during the 10 minutes.

12. The process of claim 1, wherein extracting DNA from the MRT drug substance test sample in each of the plurality of individual wells and the control test sample in each of the plurality of individual wells comprises:
centrifuging the test plate;
removing a supernatant from each well of the plurality of wells leaving a pellet at a bottom of each well of the plurality of wells;
resuspending the pellet at the bottom of each well of the plurality of wells in phosphate-buffered saline (PBS);
after resuspending the pellet at the bottom of each well of the plurality of wells in PBS, centrifuging the test plate a second time;
after centrifuging the test plate a second time, removing a second supernatant from each well of the plurality of wells leaving a pellet at a bottom of each well of the plurality of wells;
after removing the second supernatant from each well of the plurality of wells leaving a second pellet at the bottom of each well of the plurality of wells;
resuspending the second pellet at the bottom of each well of the plurality of wells in a preparation reagent;
after resuspending the second pellet at the bottom of each well of the plurality of wells, cooling the test plate in a freezer for in the range of 5 to 30 minutes;
after cooling the test plate, thermal cycling the test plate;
after thermal cycling the test plate, centrifuging the test plate a third time; and
after centrifuging the test plate a third time, removing a third supernatant from each well of the plurality of wells leaving a third pellet at a bottom of each well of the plurality of wells, wherein the third supernatant includes the DNA sample from the MRT drug substance test sample in each of the plurality of individual wells and the control test sample in each of the plurality of individual wells.

13. The process of claim 1, wherein the qPCR mixture comprises a DNA polymerase, a plurality of nucleotides, a first primer, a second primer, and molecular grade water.

14. The process of claim 1, wherein the thermocycler protocol comprises an initial denaturation step, a denaturation step, and an annealing step.

15. The process of claim 1, wherein the total CFU/mL is calculated the using the formula:
$$\text{Test Sample} \frac{CFU}{mL} = \frac{( Test Sample - Sample Background ) * \text{Dilution Factor}}{\text{Average 16s gene copy number per} \text{CFU}}$$
where Sample Background, is the average background gene copy number before CFU transformation, Test Sample is the gene copy value output by the detection system based on the standard curve, the Dilution Factor is 5.0×10⁵, and the Average 16 s gene copy number per CFU is 5.

16. A process for quantifying viable bacterial microorganisms in a microbiota restoration therapy (MRT) drug substance, the process comprising:
preparing a microbiota restoration therapy (MRT) drug substance from a human stool sample , wherein preparing the MRT drug substance comprises:
collecting a fresh stool sample from a human donor;
adding an amount of saline to the fresh stool sample;
adding polyethylene glycol to the fresh stool sample at a concentration of 30-90 g/L in saline;
mixing the fresh stool sample, saline, and polyethylene glycol together to make a mixed composition; and
filtering the mixed composition and collecting the filtrate, wherein the filtrate defines the MRT drug substance;
preparing an MRT drug substance test sample from the MRT drug substance;
preparing a control test sample from a control sample;
treating the MRT drug substance test sample and the control test sample with propidium monazide (PMA);
aliquoting a portion of the MRT drug substance test sample into a plurality of individual wells of a test plate;
aliquoting a portion of the control test sample into a plurality of individual wells of the test plate;
exposing the test plate to a light for in the range of 10 minutes extracting DNA samples from the MRT drug substance test sample in each of the plurality of individual wells and the control test sample in each of the plurality of individual wells;
diluting the DNA samples from the MRT drug substance test sample in each of the plurality of individual wells and the control test sample in each of the plurality of individual wells with molecular grade water;
adding a quantitative polymerase chain reaction (qPCR) mixture to a plurality of wells of a second test plate;
adding the diluted DNA samples from the MRT drug substance test sample in each of the plurality of individual wells and the control test sample in each of the plurality of individual wells of the test plate to the plurality of wells of the second test plate centrifuging the second test plate;
placing the second test plate in a qPCR detection system and initiating a thermocycler protocol; and
calculating the total colony forming units (CFU) per milliliter (mL) (CFU/mL) of each test sample in the plurality of individual wells of the second test plate.

17. The process of claim 16, wherein preparing the MRT drug substance test sample comprises diluting an MRT drug product with saline to form the MRT drug substance test sample.

18. The process of claim 16, wherein the light comprises a light emitting diode (LED) light.

19. The process of claim 16, wherein extracting DNA from the MRT drug substance test sample in each of the plurality of individual wells and the control test sample in each of the plurality of individual wells comprises:
centrifuging the test plate;
removing a supernatant from each well of the plurality of wells leaving a pellet at a bottom of each well of the plurality of wells;
resuspending the pellet at the bottom of each well of the plurality of wells in phosphate-buffered saline (PBS);
after resuspending the pellet at the bottom of each well of the plurality of wells in PBS, centrifuging the test plate a second time;
after centrifuging the test plate a second time, removing a second supernatant from each well of the plurality of wells leaving a pellet at a bottom of each well of the plurality of wells;
after removing the second supernatant from each well of the plurality of wells leaving a second pellet at the bottom of each well of the plurality of wells;
resuspending the second pellet at the bottom of each well of the plurality of wells in a preparation reagent;
after resuspending the second pellet at the bottom of each well of the plurality of wells, cooling the test plate in a freezer for in the range of 5 to 30 minutes;
after cooling the test plate, thermal cycling the test plate;
after thermal cycling the test plate, centrifuging the test plate a third time; and
after centrifuging the test plate a third time, removing a third supernatant from each well of the plurality of wells leaving a third pellet at a bottom of each well of the plurality of wells, wherein the third supernatant includes the DNA sample from the MRT drug substance test sample in each of the plurality of individual wells and the control test sample in each of the plurality of individual wells.

20. A process for quantifying viable bacterial microorganisms in a microbiota restoration therapy (MRT) drug substance, the process comprising:
preparing a microbiota restoration therapy (MRT) drug substance from a human stool sample , wherein preparing the MRT drug substance comprises:
collecting a human stool sample;
purifying the human stool sample to form a purified sample;
stabilizing the purified sample to form a stabilized sample;
converting the stabilized sample to a solid; and
adding one or more additives and/or excipients to the solid to form a treatment composition, wherein the treatment composition defines the MRT drug sub stance;
preparing an MRT drug substance test sample from a MRT drug substance;
preparing a control test sample from a control sample;
treating the MRT drug substance test sample and the control test sample with propidium monazide (PMA);
aliquoting a portion of the MRT drug substance test sample into a plurality of individual wells of a test plate;
aliquoting a portion of the control test sample into a plurality of individual wells of the test plate;
exposing the test plate to a light for in the range of 10 minutes extracting DNA samples from the MRT drug substance test sample in each of the plurality of individual wells and the control test sample in each of the plurality of individual wells;
diluting the DNA samples from the MRT drug substance test sample in each of the plurality of individual wells and the control test sample in each of the plurality of individual wells with molecular grade water;
adding a quantitative polymerase chain reaction (qPCR) mixture to a plurality of wells of a second test plate;
adding the diluted DNA samples from the MRT drug substance test sample in each of the plurality of individual wells and the control test sample in each of the plurality of individual wells of the test plate to the plurality of wells of the second test plate
centrifuging the second test plate;
placing the second test plate in a qPCR detection system and initiating a thermocycler protocol; and
calculating the total colony forming units (CFU) per milliliter (mL) (CFU/mL) of each test sample in the plurality of individual wells of the second test plate.


SEQUENCE LISTING (2 sequences)

SEQ ID NO: 1 - DNA, 17 bp, Artificial Sequence
  Primer JE 341F
  misc_feature (9)..(9): n is a, c, g, or t
cmtacgggnbgcascag

SEQ ID NO: 2 - DNA, 21 bp, Artificial Sequence
  Primer V3-R1
  misc_feature (7)..(7): n is a, c, g, or t
gactacnvgggtatctaatcc